(19) United States
(12) Patent Application Publication
(10) Pub. No.: US 2005/0063918 A1
(43) Pub. Date: Mar. 24, 2005

(54) Oral composition

(75) Inventors: Dominique Charmot, Campbell, CA (US); Christopher David Gibbs, Bebington (GB); Oleg Kolosov, San Jose, CA (US); Mingjun Liu, Santa Clara, CA (US); Son Hoai Nguyen, Santa Clara, CA (US); Miroslav Petro, San Jose, CA (US); Steven Paul Rannard, Bebington (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/665,710

(22) Filed: Sep. 19, 2003

(51) Int. Cl. A61K 9/68; A61K 7/16

(52) U.S. Cl. 424/48; 424/49

(57) ABSTRACT

Oral care composition comprising a polymer obtainable by copolymerising a mixture of comonomers, in which at least 40 mol % of the mixture of comonomers is constituted by a comonomer having the formula (I):
H₂C═C(R)—(X)ₙ—Y in which R is hydrogen or a methyl group, X is a divalent organic linking group, n is an integer of 0 or 1, and Y is a carboxylate or phosphonate anion, or the corresponding salt or acid thereof; and in which the balance of the mixture of comonomers is constituted by neutral and/or cationic comonomers; said composition being in the form of any one of a toothpaste, gel, foam, chewing gum, deformable strip or mouthwash and being suitable for use in the oral cavity.

The present invention relates to an oral composition comprising a polymer which is delivered to the oral surfaces during toothbrushing.

We have found that there exists a range of polymers which are delivered more effectively to the oral surfaces during brushing. Accordingly, these polymers provide a useful tool for the delivery of active substances for the treatment or prevention of oral care related conditions such as gingivitis, caries, tartar, oral malodour, etc.

Accordingly the present invention provides an oral care composition comprising a polymer obtainable by copolymerising a mixture of comonomers, in which at least 40 mol % of the mixture of comonomers is constituted by a comonomer having the formula (I):
H₂C═C(R)—(X)—Y

in which R is hydrogen or a methyl group, X is a divalent organic linking group, n is an integer of 0 or 1, and Y is a carboxylate or phosphonate anion, or the corresponding salt or acid thereof;

and in which the balance of the mixture of comonomers is constituted by neutral and/or cationic comonomers;

said composition being in the form of any one of a toothpaste, gel, foam, chewing gum, deformable strip or mouthwash and being suitable for use in the oral cavity.

In a preferred embodiment the anionic comonomer is selected from mono-2-(methacryloyl)ethyl succinate and vinyl phosphonic acid.

Further, preferred neutral and/or cationic comonomers are selected from styrene,

[2(methacryloyloxy)ethyl]trimethylammonium chloride, 2-hydroxyethylacrylate, N-[tris(hydroxymethyl)methyl]acrylamide, N-vinylpyrrolidone,

(ar-vinylbenzyl) trimethylammonium chloride, N,N-dimethylacrylamide and mixtures thereof, more preferably [2(methacryloyloxy)ethyl]trimethylammonium chloride, N-vinylpyrrolidone, N-[tris(hydroxymethyl)methyl] acrylamide, N,N-dimethylacrylamide, (ar-vinylbenzyl) trimethylammonium chloride and mixtures thereof.

More preferred polymers include those obtainable by copolymerising a mixture of vinyl phosphonic acid, [2(methacryloyloxy)ethyl]trimethylammonium chloride and 2-hydroxyethylacrylate.

Further more preferred polymers include those obtainable by copolymerising a mixture of vinyl phosphonic acid and N-[tris(hydroxymethyl)methyl] acrylamide.

Of these preferable polymers the most preferred polymers include the following mixtures of comonomers of Formula (I) and neutral and/or anionic comonomers:
(a) where the comonomer of Formula (I) is vinyl phosphonic acid and the further comonomer
[2(methacryloyloxy)ethyl]trimethylammonium chloride and/or 2-hydroxyethylacrylate. Preferably the vinyl phosphonic acid is present in a mol % ratio in the polymerisation mixture of from 20 to 80, more preferably from 40 to 70 and most preferably about 60%. Where [2(methacryloyloxy)ethyl]trimethylammonium chloride is present it is preferably present in from 5 to 40, more preferably from 10 to 20 and most preferably at 20 mol % of the polymerisation mixture. Where 2-hydroxyethylacrylate is present it is preferably present in from 5 to 40, more preferably from 10 to 20 and most preferably at 20 mol % of the polymerisation mixture. Especially preferred polymers of this comonomer combination type include those with a mol % ratio of around 60:20:20 of is vinyl phosphonic acid: [2(methacryloyloxy)ethyl]trimethylammonium chloride: 2-hydroxyethylacrylate in the copolymerisation mixture.
(b) where the comonomer of Formula (I) is vinyl phosphonic acid and the further comonomer is selected from N-[tris(hydroxymethyl)methyl] acrylamide, N,N-dimethylacrylamide, N-vinylpyrrolidone and mixtures thereof and the VPA is present at from 40 to 90 mol % of the polymerisation mixture, more preferably from 45 to 80%. The remainder preferably being just one of the proposed selected monomers and making up the remainder of the polymerisation mixture.

The polymer according to Formula (I) is preferably present at from 0.01 to 10% by weight of the composition. Preferably, in an amount ranging from 0.05 to 5% by weight of the composition.

Preferably the polymer according to the invention is substantially anionic overall.

Preferably, the polymer includes a random sequence of monomer units.

The composition according to the invention may also comprise a halogenated hydroxydiphenyl ether compound, more preferably 2′,4,4′-trichloro-2-hydroxy-diphenyl ether, hereinafter known as triclosan. Preferably the halogenated hydroxydiphenyl ether is present at from 0.01 to 0.5% by weight of the composition. A further preferred group of antimicrobial substances are the parahydroxybenzoic acid esters, also known as parabens, and their structural analogues. Preferred parabens are the medium chain length parabens such as hexyl, heptyl, octyl, nonyl and decyl parabens. Most preferred is the n-octyl paraben.

The composition according to the invention may also comprise a divalent metal salt. Preferably, the divalent metal salt is a salt selected from the group consisting of zinc- and stannous salts such as zinc citrate, zinc sulphate, zinc glycinate, sodium zinc citrate, stannous pyrophosphate and mixtures thereof. The preferable divalent metal salt is zinc citrate.

Suitably, the amount of divalent metal salt ranges from 0.01 to 10% by weight of the composition, preferably from 0.05 to 5% by weight, more preferably from 0.1 to 2% by weight and especially preferably from 0.3 to 0.9% by weight of the composition.

The oral composition according to the invention comprise further ingredients which are common in the art, such as:

antimicrobial agents, e.g. chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition. Preferred abrasives are chalk and silica, more preferably fine ground natural chalk.

Humectants such as glycerol, sorbitol, propyleneglycol, xylitol, lactitol etc.;

binders and thickeners such as sodium carboxymethyl-cellulose, hydroxyethyl cellulose (Natrosol®), xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

The polymer according to the invention is capable of delivering itself to the oral surfaces during brushing. Preferably, in conjunction with a benefit agent selected from any of those included herein. Most preferable of these benefit agents are the antimicrobials, anti-caries agents, anti-tartar agents, anti-malodour agents and bleaching or tooth whitening agents.

In a second aspect the present invention provides a process for preparing an oral care composition according to any one of claims 1 to 5, comprising the steps of:

preparing a mixture of comonomers as defined in the first aspect of the invention in an ethanol/water diluent;

polymerising the mixture by heating it under inert gas in the presence of an initiator;

extracting the polymer so obtained and blending it with one or more oral care actives and/or excipients so as to produce an oral care composition which is in the form of any one of a toothpaste, gel, foam, chewing gum, deformable strip or mouthwash and which is suitable for use in the oral cavity.

Preferably, the monomers are mixed at about 20% by (w/v) in ethanol:water mixture of from 50:50 to 95:5, more preferably from 70:30 to 90:10 and most preferably 80:20.

Preferably, the initiator is AIBN and is added at from 0.1 to 5%, preferably from 0.5 to 2.0% and most preferably at 1.0% mol with respect to the total monmomers.

Preferably, the inert gas is argon.

Preferably, the heating step involves heating for up to 36, preferably up to 24 and most preferably for 18 hours at above 45° C., prefereably more than 50° C. and most preferably at aboput 65° C.

The monomer mixture is then preferably cooled to room temperature.

The polymer is then preferably, diluted with ethanol:water of from 50:50 to 95:5, more preferably from 70:30 to 90:10 and most preferably 80:20 to bring the final concentration to about 10% (w/v).

Preferably the reaction is carried out in a well of a 96-well plate.

### EXAMPLES

Manufacture of Polymers

Manufacture of polymers is done by preparing a mixture of comonomers as defined in any one of claims 1 to 5 in an ethanol/water diluent and polymerising the mixture by heating it under inert gas in the presence of an initiator

Assessment of Delivery to Oral Surfaces:

The following polymers were used as control polymers throughout the examples:
1) Pluronic polymer F 127, a polyethyleneoxide-b-polypropyleneoxide-b-polyethyleneoxide triblock copolymer having a total molecular weight (M_{W}) of about 12,600 and containing about 70 wt. % polyethyleneoxide units;
2) Gantrez polymer AN-119, a PMA-VE copolymer having a molecular weight (Mₙ) of about 80,000; and
3) C6 and C12 Gantrez derivatives made by the applicant. The Gantrez polymer described above (#2) was reacted with hexylamine and dodecylamine.

### Example 1

The control polymers were labeled by fluorescein and dissolved in deionized water under stirring to make up stock solutions having a polymer concentration of 80 g/l.

These stock solutions were then diluted (dilution ratio: 40:1) with an artificial saliva composition in order to prepare control polymer formulations in saliva having a polymer concentration of 2 g/l, followed by filtration. The artificial saliva composition was made up according to the method described in Wong, L and Sissons, C H; Archives of Oral Biology 46 (2001 477-486, A comparison of human dental plaque microcosm biofilms grown in an undefined medium and a chemically defined artificial saliva.

Moreover, an artificial saliva composition containing the free dye was prepared.

Additional formulations containing SDS (sodium dodecyl sulfate) were prepared in order to study the effect of a surfactant.

Pig tongue was selected as a control model substrate for soft oral tissue, representing human tongue, gums, etc. The model substrate was pre-treated with a saliva composition overnight. The pre-treated substrate was spotted by the control polymer formulations (500 μl per spot), followed by washing out non-adsorbed polymer by saliva. The pre-treated substrate was also spotted by the saliva formulation containing the free dye.

HAP powder (porous HAP particles having a size of about 20 μm) and HAP discs (discs size: 0.5 inch DIA×0.03 inch×0.05 inch) were selected as model substrates for hard oral tissue, representing the enamel of the human teeth. 50 mg of HAP was put into 800 μl vials (0.45 μm PP filter, UNIFILTER from Whatman). Next, 600 μl of saliva was added to each vial and the HAP suspension was shaked/stirred at least three hours, followed by filtering and drying by air. The substrate was then exposed to the polymer formulations, followed by washing out non-adsorbed polymer by saliva. The substrate was also exposed to the saliva formulation containing the free dye.

The control polymer formulations as well as the artificial saliva formulations containing the free dye were screened for adsorption on both soft and hard oral tissues by using a fluorescence imaging system.

### Example 2

This example demonstrates the screening of polymers for adsorptivity to both hard and soft oral tissues.

The relevant monomers were used for the preparation of polymers. Various homopolymers and copolymers obtained by polymerizing the monomers were labeled by fluorescein and dissolved in deionized water under stirring to make up stock solutions having a polymer concentration of 80 g/l. These stock solutions were diluted (dilution ratio: 40:1) with an artificial saliva composition in order to prepare polymer formulations in saliva having a polymer concentration of 2 g/l, followed by filtration.

Soft and hard oral tissues (pig tongue and HAP powder/discs) were exposed to the polymer formulations in the same manner as in Example 1, and the obtained polymers were screened for adsorption on both soft and hard oral tissues as in Example 1, always accompanied by a control polymer (Pluronic polymer) in order to normalize the response.

TABLE

| | Chemistry | | | | | | | |
|---|---|---|---|---|---|---|---|---|
| | Monomer 1 | | Monomer 2 | | Monomer 3 | | | |
| | Name | % | Name | % | Name | % | A | B |
| 1 | VBTMAC | 60 | Sty | 20 | THMMAM | 20 | 4.3 | 7.8 |
| 2 | VBTMAC | 60 | Sty | 20 | VPL | 20 | 6.0 | 5.8 |
| 3 | DMAPMAM | 60 | MAES | 20 | THMMAM | 20 | 5.2 | 5.0 |
| 4 | DMAPMAM | 60 | MAES | 20 | BA | 20 | 4.8 | 5.4 |
| 5 | DMAPMAM | 90 | VA | 10 | | | 4.1 | 6.1 |
| 6 | VBTMAC | 25 | DMA | 75 | | | 3.1 | 7.1 |
| 7 | VBTMAC | 60 | EHA | 20 | HEA | 20 | 4.6 | 5.1 |
| 8 | MAETMAC | 60 | MAES | 20 | EHA | 20 | 6.3 | 3.3 |
| 9 | DMAPMAM | 75 | DMA | 25 | | | 5.2 | 4.3 |
| 10 | DMAPMAM | 60 | EHA | 20 | PEGMA | 20 | 4.6 | 4.5 |
| 11 | VBTMAC | 50 | VPA | 50 | | | 3.0 | 6.9 |
| 12 | MAETMAC | 90 | AA | 10 | | | 4.2 | 4.7 |
| 13 | DMAPMAM | 33 | VA | 33 | VPL | 33 | 2.7 | 6.8 |
| 14 | DMAPMAM | 60 | AMMPSA | 20 | PEGMEMA | 20 | 4.8 | 3.8 |
| 15 | VBTMAC | 60 | AA | 20 | EHA | 20 | 3.4 | 3.2 |
| 16 | DMAPMAM | 10 | THMMAM | 90 | | | 2.2 | 3.8 |
| 17 | AEMAH | 75 | DMA | 25 | | | 2.4 | 3.0 |
| 18 | DMAPMAM | 60 | AA | 20 | VPL | 20 | 2.3 | 3.5 |
| 19 | MAETMAC | 20 | VPA | 60 | HEA | 20 | 0.2 | 4.4 |
| 20 | VPA | 75 | THMMAM | 25 | | | 0.4 | 4.1 |
| 21 | VPA | 50 | THMMAM | 50 | | | 0.1 | 2.4 |
| 22 | MAETMAC | 20 | MAES | 60 | Sty | 20 | 0.1 | 2.3 |
| 23 | VPA | 50 | VPL | 50 | | | 0.5 | 2.6 |
| 24 | VBTMAC | 50 | MAES | 50 | | | 0.4 | 2.0 |
| 25 | VPA | 75 | DMA | 25 | | | 0.6 | 2.2 |
| 26 | HEA | 100 | | | | | 0.0 | 0.0 |
| 27 | DMA | 100 | | | | | 0.0 | 0.1 |
| 28 | THMMAM | 100 | | | | | 0.0 | 0.1 |
| 29 | VPL | 100 | | | | | 0.0 | 0.0 |
| 30 | DMAEA | 0 | THMMAM | 100 | | | 0.0 | 0.5 |
| 31 | DMAEA | 0 | HEA | 100 | | | 0.0 | 0.5 |
| 32 | VA | 10 | DMA | 90 | | | 0.0 | 0.5 |
| 33 | VA | 10 | THMMAM | 90 | | | 0.0 | 0.5 |

A is delivery to soft surfaces.
B is delivery to hard surfaces.
VBTMAC is (ar-vinylbenzyl) trimethylammonium chloride
DMAPMAM is (dimethylaminopropyl) methacrylamide
MAETMAC is [2(methacryloyloxy) ethyl]trimethylammonium chloride
AEMAH is 2-aminoethylmethacrylate hydrochloride
STY is styrene
MAES is mono-2-(methacryloyl)ethyl succinate
VA is vinyl acetate
DMA is N,N-dimethylacrylamide
EHA is 2-ethylhexylacrylate
VPA is vinylphosphonic acid
AA is acrylic acid
AMMPSA is 2-acrylamido-2-methyl-1-propanesulfonic acid
THMMAM is N-[tris (hydroxymethyl)methyl] acrylamide
VPL is N-vinylpyrrolidone
BA is butyl acrylate
HEA is 2-hydroxyethylacrylate
PEGMEMA is polyethyleneglycol methylethermethacrylate
DMAEA is

1 Oral care composition comprising a polymer obtainable by copolymerising a mixture of comonomers, in which at least 40 mol % of the mixture of comonomers is constituted by a comonomer having the formula (I):
H₂C═C(R)—(X)ₙ—Y
in which R is hydrogen or a methyl group, X is a divalent organic linking group, n is an integer of 0 or 1, and Y is a carboxylate or phosphonate anion, or the corresponding salt or acid thereof;
and in which the balance of the mixture of comonomers is constituted by neutral and/or cationic comonomers;
said composition being in the form of any one of a toothpaste, gel, foam, chewing gum, deformable strip or mouthwash and being suitable for use in the oral cavity.

2 Oral care composition according to claim 1, in which the anionic comonomer is selected from mono-2-(methacryloyl)ethyl succinate and vinyl phosphonic acid.

3 Oral care composition according to claim 2, in which the neutral and/or cationic comonomers are selected from styrene, [2(methacryloyloxy)ethyl]trimethylammonium chloride, 2-hydroxyethylacrylate, N-[tris(hydroxymethyl)methyl] acrylamide, N-vinylpyrrolidone,
(ar-vinylbenzyl) trimethylammonium chloride,
N,N-dimethylacrylamide and mixtures thereof.

4 Oral care composition according to claim 3, comprising a polymer obtainable by copolymerising a mixture of vinyl phosphonic acid, [2(methacryloyloxy)ethyl]trimethylammonium chloride and 2-hydroxyethylacrylate.

5 Oral care composition according to claim 3, comprising a polymer obtainable by copolymerising a mixture of vinyl phosphonic acid and N-[tris(hydroxymethyl)methyl] acrylamide.

6 Process for preparing an oral care composition according to claim 1, comprising the steps of:
preparing a mixture of comonomers in an ethanol/water diluent;
polymerising the mixture by heating it under inert gas in the presence of an initiator;
extracting the polymer so obtained and blending it with one or more oral care actives and/or excipients so as to produce an oral care composition which is in the form of any one of a toothpaste, gel, foam, chewing gum, deformable strip or mouthwash and which is suitable for use in the oral cavity.
